(19) United States
(12) Patent Application Publication
(10) Pub. No.: US 2011/0123463 A1
(43) Pub. Date: May 26, 2011

(54) TOPICAL COMPOSITIONS

(75) Inventors: Jo Klaveness, Oslo (NO); Pal Rongved, Oslo (NO)

(73) Assignee: OMEGATRIAS

(21) Appl. No.: 12/991,742

(22) PCT Filed: May 11, 2009
(86) PCT No.: PCT/GB09/01187

§ 371 Date: Jan. 31, 2011

(30) Foreign Application Priority Data
May 9, 2008 (GB) 0808479.0

(51) Int. Cl. A61K 8/36; A61K 31/20; A61Q 19/00; A61Q 11/00; A61Q 19/04

(52) U.S. Cl. 424/49; 514/560; 424/59

(57) ABSTRACT

A topical composition, e.g. pharmaceutical or cosmetic composition comprising at least one cyclodextrin complex of a marine unsaturated fatty acid or derivative thereof wherein the weight ratio of cyclodextrin to marine fatty acid or derivative thereof is between 1:10 and 10:1.

This invention relates to topical compositions, e.g. pharmaceutical and cosmetic compositions for topical application to the skin, comprising one or more marine unsaturated fatty acids and/or fatty acid derivatives in the form of cyclodextrin complexes and the use of such dermal formulations for enhancement of skin properties or treatment or prophylaxis of disorders related to skin. In particular, the invention concerns the use of stable and effective topical compositions comprising cyclodextrin encapsulated omega-3 fatty acids or derivatives thereof for cosmetic use for enhancing skin properties or in the treatment or prevention of skin diseases.

Omega-3 and omega-6 fatty acids are fatty acids essential to human health but ones which cannot be manufactured by the body. There are several major types of unsaturated fatty acids that are ingested in foods and used by the body: the omega-6 fatty acid arachidonic acid, and the omega-3 fatty acids alpha-linolenic acid (ALA), eicosapentaenoic acid (EPA) and docosahexaenoic acid (DHA). Once eaten, the body may convert ALA to EPA and DHA, but the capacity for this conversion is very limited, so therefore dietary supply of EPA and DHA is needed. The major source of omega-3 fatty acids is marine oils. It is important to maintain an appropriate balance of omega-3 and omega-6 in the diet as these two substances work together to promote health.

Extensive research indicates that whereas omega-6 has a role in promoting inflammation, omega-3 fatty acids reduce inflammation and help prevent certain chronic diseases such as heart disease and arthritis. Especially DHA is highly concentrated in the brain and appears to be particularly important for cognitive and behavioural function. In fact, infants who do not get enough omega-3 fatty acids from their mothers during pregnancy are at risk of developing vision and nerve problems.

An inappropriate balance of these essential fatty acids contributes to the development of disease while a proper balance helps maintain and even improve health. A healthy diet should consist of roughly one to two times more omega-6 fatty acids than omega-3 fatty acids.

With the development of convenience foods and a general decline in the consumption of healthy foodstuffs such as fresh fish, fruit and vegetables, the typical American diet tends to contain 11 to 30 times more omega-6 fatty acids than omega-3 fatty acids and many researchers believe this imbalance is a significant factor in the rising rate of inflammatory disorders in the United States.

In contrast, however, the Mediterranean diet consists of a healthier balance between omega-3 and omega-6 fatty acids and many studies have shown that people who follow this diet are less likely to develop heart disease. The Mediterranean diet does not include much meat (which, dependent on the feed of the animal, might be high in omega-6 fatty acids) and emphasizes foods rich in omega-3 fatty acids including whole grains, fresh fruits and vegetables, fish, olive oil, garlic, as well as moderate wine consumption.

Thus, since their discovery in the 1970s, and the finding that the ratio of omega-3 to omega-6 acids is imbalanced in the diets of many individuals, omega-3 fatty acids or their derivatives have been made available to consumers as dietary supplements to try to restore the desired omega-3 to omega-6 balance. Omega-3 fatty acids or derivatives thereof are thus now taken routinely by many hundreds of thousands of individuals to prevent a variety of illnesses such as arthritis, cardiac infarction and stroke.

Omega-3 fatty acids are often provided to consumers in their naturally occurring triglyceride form. The omega-3 fatty acid triglyceride or the free fatty acid itself are generally sourced from natural oils such as marine oils. Since these substances are prone to oxidation, the marine oils easily go rancid leading to the unpleasant fishy after taste of omega-3 supplements, which the consumer dislikes. It is also a major problem for many individuals, such as the elderly and children, to swallow the gelatine capsules used today to contain the omega-3 material. Capsules are also expensive to prepare. It would be useful therefore if omega-3 compounds could be offered in alternative dosage forms.

The present invention concerns compositions designed to enhance skin appearance or treat or prevent skin disorders. It is accepted that omega-3 administered orally has a positive effect on the skin. However, there are relatively few publications describing oral formulations specifically for improvement of the skin in which omega-3 fatty acid compounds are used. WO2007/116052 (Hindustan Unilever) describes oral compositions comprising docosahexaenoic acid (DHA) and genistein for enhancing skin properties, in particular to prevent ageing.

The related WO2007/115934 (Hindustan Unilever) application also describes oral compositions comprising DHA or eicosapentaenoic acid (EPA) and other components which allegedly have an anti-ageing effect on the skin.

WO2006/056293 (Hindusan Lever et al) describes an oral emulsion comprising EPA and DHA for enhancing the skin properties.

WO2007/112996 (DSM) describes the use of various unrelated compounds including some omega-3 fatty acids for improving the physical appearance of the skin, e.g. for reducing cellulite. For the most part, the active components are suggested for incorporation in dietary supplements in this application.

As noted above, in view of the relatively high dosages of fatty acid compounds required to cause a physiological effect and in view of the problems of formulating sufficient fatty acid compound into a suitable oral dosage form, the present inventors have sought alternative modes of administration of the active component.

The present invention concerns compositions for topical application as opposed to oral administration. There are, however, even fewer documents describing topical compositions containing omega-3 fatty acids.

WO2007/015027 (L'Oreal) describes cosmetic and/or dermatological compositions for prevention and/or treatment of sensitive or dry skin comprising a probiotic microorganism in combination with at least one polyunsaturated fatty acid or derivative thereof.

WO2008/000974 (L'Occitane) describes cosmetic formulations based on polyunsaturated fatty acids and a variety of other components. These compositions are designed to stimulate “microcirculation” and impart “dynamism” to cells.

U.S. Pat. No. 4,710,383 (Dick) describes dermal compositions comprising essential fatty acids for transdermal delivery.

A problem with these formulations and compositions of fatty acid compounds in general is their stability. Naturally occurring fatty acid compounds typically contain a polyunsaturated backbone which is susceptible to oxidation and hence degradation.

The problem of degradation is however exacerbated in a topical formulation as the composition is inherently exposed to oxidative agents as it is applied on the skin. Light is a major contributor to oxidative degradation of these molecules' and topical compositions are inherently exposed to light. Another factor is heat and even the natural heat of the body encourages degradation.

Perhaps more significantly, there is a shelf-life issue which is more significant with a topical composition than other administration routes. An oral composition can be stored in an airtight environment, e.g. in a special blister pack or the like and can be kept away from light and air until it is ready for consumption. Only when the dosage form is to be taken is it exposed to these elements however the dosage form is swallowed rapidly thereafter. As each dosage form is separately packaged, consumption of one tablet or capsule does not encourage degradation of other dosage forms.

In contrast, a topical composition, in particular a cosmetic composition is often opened, used and then replaced on the bathroom shelf. Whilst the contents of the container may well start off being protected from the air in a sealed container, on opening of the package for the first time, air is allowed in. Unlike an oral form however, a topical composition is likely to come in a container where there is enough active material for repeated applications. As each day passes from initial opening, degradation can occur and thus the problem of decay is acute in topical compositions.

One solution to the stability problem has been to complex the polyunsaturated fatty acids with a compound such as cyclodextrin. EP-A-470452 (Staroil) describes methods for the preparation of polyunsaturated fatty acid complexes with cyclodextrins in aqueous solution.

U.S. Pat. No. 4,438,106 (Kureaha) describes preparation of cyclodextrin EPA and DHA complexes. These complexes are prepared using large excess of cyclodextrins.

WO00/53637 (Commissariat a L energie atomique) describes fatty acid complexes with gamma-cyclodextrin.

U.S. Pat. No. 6,025,510 (Wacker-Chemie) describes stabilization of vegetable oils comprising polyunsaturated fatty acid radicals using gamma-cyclodextrin.

Fatty acid complexes between fatty acids and derivatives thereof with cyclodextrins are thus described in the prior art, however, the weight ratio between cyclodextrin and fatty acid is high. These prior art disclosures thus require a considerable excess of cyclodextrin to be present and hence a limited amount of the desired fatty acid material. Since the amount of fatty acid required in order to attain a pharmacologically active quantity of active ingredient is high, this means that regular doses of the fatty acid need to be administered. Moreover, it makes the formulation expensive as large quantities of cyclodextrin need to be employed.

Surprisingly, we have now found that unsaturated fatty acids or derivatives thereof derived from marine sources (from hereon marine fatty acids or derivatives thereof), preferably unsaturated fatty acid esters or unsaturated fatty acid phospholipids and especially marine unsaturated fatty acid triglycerides and most especially those comprising EPA and DHA or derivatives thereof, in the form of complexes with cyclodextrins can be prepared as stable solid materials and can be formulated into topical compositions with high concentrations of the fatty acid component(s) in the composition.

The present inventors have realised that stable topical formulations can be prepared from solid unsaturated fatty acid compound cyclodextrin complexes, where the complex comprises high amounts of an unsaturated fatty acid component.

Thus, viewed from one aspect of the present invention provides a topical composition, e.g. pharmaceutical or cosmetic composition, comprising at least one cyclodextrin complex of a marine unsaturated fatty acid or derivative thereof, preferably comprising one or both of EPA and DHA or derivative(s) thereof, wherein the weight ratio of cyclodextrin to marine fatty acid or derivative thereof is preferably between 1:10 and 10:1.

Viewed from another aspect the invention provides a topical composition comprising at least one cyclodextrin complex of a marine unsaturated fatty acid or derivative thereof, preferably comprising one or both of EPA and DI-IA or derivative(s) thereof, wherein the weight ratio of cyclodextrin to fatty acid or derivative thereof is preferably between 1:10 and 10:1 for enhancing skin properties or for the treatment or prophylaxis of skin diseases.

Viewed from another aspect the invention provides use of at least one cyclodextrin complex of an unsaturated fatty acid or derivative thereof wherein the weight ratio of cyclodextrin to fatty acid or derivative thereof is preferably between 1:10 and 10:1 in the manufacture of a medicament for enhancing skin properties and/or in the treatment or prophylaxis of skin diseases.

Viewed from another aspect the invention provides a method of enhancing skin properties comprising topically applying to the skin of a patient a topical composition as hereinbefore described.

Viewed from another aspect the invention provides a method of treatment or prophylaxis of skin diseases comprising applying to the skin a patient a topical composition as hereinbefore described.

Viewed from another aspect the invention provides a process for the manufacture of a topical composition as hereinbefore defined comprising forming a cyclodextrin complex with an unsaturated fatty acid or derivative thereof using techniques well known in the prior art and preparing a topical composition thereof.

The formation of a topical composition comprising a cyclodextrin complex of an omega-3 fatty acid is also new and forms a further aspect of the invention.

Viewed from another aspect therefore the present invention provides a topical composition, e.g. pharmaceutical or cosmetic composition, comprising at least one cyclodextrin complex of an omega-3 fatty acid or derivative thereof.

For the avoidance of doubt, the ratio of cyclodextrin to fatty acid or derivative thereof in the above statements of invention refers to the total amount of marine fatty acid(s) or derivative(s) thereof present and not just the amount of the unsaturated fatty acid or derivative thereof. The topical compositions of the invention may contain a variety of fatty acids (or only one) and the ratio refers to the total fatty acid or derivative thereof content coming from a marine source. Thus, where the marine fatty acid source also contains a saturated fatty acid component this would be taken into account when calculating the ratio of cyclodextrin to fatty acid compound.

The compositions of the invention are for topical use. A topical medicine is one which is applied to a body surface such as the skin or mucous membranes. Ideally, the compositions of the invention are for administration on the skin. The active components of the invention are preferably absorbed through the skin, i.e. are transdermal.

In the description which follows, the term “fatty acid compound” is used to cover a fatty acid per se or a derivative thereof.

The at least one fatty acid compound present in the topical compositions of the invention is unsaturated, especially polyunsaturated. Most preferably, the topical compositions of the invention comprise at least one omega-3 fatty acid compound.

Any fatty acid compound, preferably an omega-3 fatty acid compound, present in the topical compositions of the invention can preferably be derived from a marine source. Marine oils which contain fatty acids, typically present as esters of the fatty acids, are well known in the art. Highly preferably, the source of the fatty acid compound is a marine oil such as a fish oil or krill oil. Crude marine oil used in this invention can be derived from any marine source such as fish, especially seawater fish such as tuna, sardines, salmon, mackerel, herring, trout, halibut, cod, haddock, catfish, sole etc. The use of oily fish is preferred. Cod liver oil and salmon oil are the especially preferred sources of the desired fatty acid compounds.

The fatty acid compounds can also derive from marine mammals such as seals, walrus or sea lions, preferably seals. Seal oil has been found to be especially rich in omega-3 fatty acid compounds, e.g. of the order of 20-25 wt %.

In a highly preferred embodiment therefore, the invention utilises krill oil, salmon oil or cod liver oil as a source of fatty acid compounds. These oils are available from a variety of commercial sources. In a further highly preferred embodiment, these oils can be used in the invention without any specific refinement steps. Thus, for example, the cod liver oil used in the invention can be complexed with cyclodextrin in its native form.

Preferably the krill oil used herein is the lipid fraction of krill, e.g. as described in WO2007080515.

The composition of the present invention might also comprise plant or animal oils in addition to the marine oils. The plant and/or animal oils optionally present in the composition of the invention are preferably not in the form of a cyclodextrin complex. Suitable plant oils include rapeseed oil, corn oil, soya oil, sunflower oil, vegetable oil and olive oil. Animal oils include tallow oil. As these oils are not marine oils, if they are present they do not contribute to the ratio of cyclodextrin to fatty acid compound.

The topical compositions of the invention can contain one unsaturated fatty acid compound or a mixture of unsaturated fatty acid compounds. Preferably, they will contain a mixture of fatty acid compounds, especially unsaturated fatty acid compounds, especially a mixture of polyunsaturated fatty acid compounds. It will be appreciated that the topical compositions of the invention might also contain saturated fatty acid compounds as these are also present in naturally occurring unsaturated fatty acid compound sources. Ideally, the topical compositions of the invention will contain a plurality of unsaturated fatty acid compounds.

An unsaturated fatty acid compound contains one or more carbon carbon double bonds in the carbon backbone; Preferably, the carbon backbone is polyunsaturated. Preferably, at least one fatty acid compound is an omega-3 fatty acid compound in which the double bond most distant from the carboxylic acid functionality is located at the third bond counted from the end (omega) of the carbon chain. The fatty acid compound may also be an omega-6 fatty acid compound where the double bond most distant from the carboxylic acid functionality is located at the sixth bond counted from the end (omega) of the carbon chain. A composition of the invention most preferably contains a variety of omega-3 and omega-6 fatty acid compounds.

The total concentration of omega-3 fatty acid compounds in a crude oil varies depending on the natural source in question but, for example, in sea fish, the amount of the omega-3 compounds is approximately 25 wt %.

An unsaturated fatty acid compound which can form part of the tablet of the invention may be those of formula (I):

CH₃(CH₂)ₙ—(CH=CH═CH₂)ₘ—(CH₂)_{S}—COOH (I)

wherein n, m and s are integers, e.g. of 1 to 10;

or a derivative thereof.

Subscript n is preferably 1. Subscript m is preferably 2 to 8. Subscript s is preferably 1 to 6. Ideally, the carbon chain is linear although it is within the scope of the invention for the backbone to carry alkyl side chains such as methyl or ethyl. (For this formula DHA n=1, m=6 and s=1, for EPA n=1, m=5 and s=1. In ALA, n=4, m=2 and s=6)

Omega-3 fatty acid compounds of use in the compositions of the invention are preferably those which contain at least 18 carbon atoms in the carbon backbone. Lower chain fatty acids (those of 17 carbon atoms or less in the backbone) appear to show fewer useful therapeutic effects, but can be useful in applications like fish or animal feed.

Thus, a preferred unsaturated fatty acid compound is one of formula (I′)

CH₃CH₂CH=CH—R—COOH (I′)

wherein R is a C₁₃₊ alkylene group (e.g. C₁₃₋₂₅) optionally containing 1 or more double bonds, preferably non-conjugated;

or a derivative thereof.

Ideally, the R group is linear although it is within the scope of the invention for the backbone to carry alkyl side chains such as methyl or ethyl. The total number of carbon atoms in the chain is preferably 16 to 22. Moreover, R is preferably 13, 15, 17, 19 etc. i.e. the number of carbon atoms in the chain is preferably even. Whilst it will be appreciated that the omega-3 enriched compositions of the invention will, most likely, contain a variety of different omega-3 based compounds, a highly preferred compound of formula (I) is eicosapentaenoic acid (EPA) or docosahexaenoic acid (DHA) or a derivative thereof, e.g. triglyceride, phospholipid, sodium salt or polyamino alcohol salt thereof.

In a highly preferred embodiment, the fatty acid compounds comprise a mixture of DHA and EPA or derivatives thereof. The weight ratio between said components (in terms of complex weight) may be in the range 1:9 to 9:1 wt %, preferably 30:70 to 70:30, more preferably 40:60 to 60:40 EPA complex/DHA complex.

The topical compositions of the invention may also contain omega-6 fatty acids. Preferred omega-6 fatty acids are those of formula (II):

CH₃CH₂CH₂CH₂CH₂CH═CH—R″—COOH (II)

wherein R″ is a C₅₊ alkylene group (e.g. C₁₀₋₂₂) optionally containing 1 or more double bonds;

or derivatives thereof.

Ideally, the R″ group is linear although it is within the scope of the invention for the backbone to carry alkyl side chains such as methyl or ethyl.

The number of carbon atoms in R″ is preferably 10, 12, 14, 16 etc, i.e. the number of carbon atoms in the chain is preferably even. In a preferred embodiment the omega-6 fatty acid compound is ALA, gamma-linolenic acid (GLA) or conjugated linoleic acid (CLA), or a derivative thereof, e.g. a triglyceride, phospholipid, sodium salt or polyamino alcohol salt thereof.

Whilst it will be appreciated that the topical compositions of the invention will, most likely, contain a variety of different omega 3 and 6 based compounds, highly preferred compounds of formula (II) are C18, C20 and C22 compounds.

Preferably, the fatty acid compounds of the invention will have at least 10 carbon atoms, e.g. at least 12 carbon atoms, such as at least 14 carbon atoms in the fatty acid portion of the molecule, i.e. a fatty acid compound must comprise at least 10 carbon atoms.

Ideally compounds of formula (I), (I′) or (II) will be multiply unsaturated, e.g. contain 2 to 10 double bonds, especially 4 to 7 double bonds. Preferably double bonds are not conjugated either to each other or to the carbonyl functionality.

At least one, e.g. 2 or 3, preferably all double bonds are preferably in the cis configuration.

Crude oils contain a variety of fatty acids or derivatives thereof (e.g. esters thereof, in particular triglycerides) having differing carbon chain lengths and differing levels of unsaturation. Of course not all these fatty acids will be omega-3 unsaturated fatty acid compounds, some will be omega-6 unsaturated, some may be saturated oils. Topical formulations comprising a mixture of these fatty acid compounds are therefore covered.

Whatever the nature of the fatty acid material, it is readily available from commercial sources. Pure DHA and EPA can be purchased and, if desired, converted to an appropriate derivative using trivial chemistry.

In a preferred embodiment, the topical composition of the invention contains only unsaturated fatty acid components and is therefore free of saturated fatty acids or derivatives thereof.

By derivative of a fatty acid, e.g. omega-3 or omega-6 fatty acid, is meant a salt, amide or ester thereof, or any other compound where the COOH group is functionalised in such a way that it will return to a COOH group upon treatment, e.g. upon hydrolysis, e.g. a phospholipid thereof. Typically, the fatty acid compounds in the compositions of the invention are in the form of esters, e.g. C₁₋₁₂-alkyl esters, especially C₁₋₄ esters such as methyl, ethyl and propyl esters, or more especially glycerides, in particular triglycerides, i.e. the fatty acid derivative is a triglyceride.

Preferred salts are those of alkali metals, e.g. sodium, or ammonium salts. Highly preferred salts include those formed with amino alcohol compounds in particular polyamino alcohol salts. The amino alcohol compound required to allow formation of such salts is preferably a hydroxylated amine with the general formula

((HO(R′)_{0/1})ₙ)R₁NHR₂)ₘ (III)

where n is an integer from 3 to 6, R′ is methyl, R₁ is a C1-20 alkylene group, R₂ is H or an C₁₋₆alkyl side chain, preferably methyl and m is an integer from 1 to 20.

In the case of n=3, R′ is present and is methyl, R₁ is methyl and R₂=H, the hydroxyamine may be tris-(hydroxymethyl)-methylamine (Tris). In the case of n=5, R′ is absent, R₁ is a straight six carbon chain and R₂ is Me and m=1, the hydroxyamine may be meglumine.

Polyamino alcohol salts of the fatty acids are formed using polyamino alcohol compounds. Polyamino compounds are those containing two or more amino groups available for forming an ammonium salt with the carboxyl group of the fatty acid. Ideally, the polyamino compound employed will be a polyamino sugar. Such polyaminosugars will preferably contain a plurality of saccharide units along with a plurality of hydroxyl groups to assist the solubility of the formed salt.

Suitable polyaminosugars may be derived from chitin, especially chitosan. A suitable polyamino sugar may have a general formula

(C₆H₁₄NO₅)ₚ (IV)

where p is an integer of 2 or more, e.g. 5 to 15.

Especially preferred polyamino sugars are those available from FMC or Novamatrix. Chitosan is often supplied in acetate form, i.e. the amine groups are protected. The acetate can be removed using known ion exchange techniques to release the free polyamino form of chitosan.

Mixtures of derivatives and/or acids may also be present in the topical compositions of the invention.

In a further highly preferred embodiment the fatty acid compounds are formed from fatty acid residues with phospholipids. Omega-3/phospholipid emulsions are described in U.S. Pat. No. 5,434,183 as dietary compositions. These emulsions are however, ideal for complexation with cyclodextrin and hence for use in the topical compositions of the present invention.

In the most preferred embodiment, the invention provides topical compositions comprising at least EPA and DHA in the form of free acids, physiologically acceptable salts, ethyl esters, phospholipids or triglycerides thereof. Where a derivative of EPA or DHA is employed, it is preferred if the derivative is in the form of an ester, especially a triglyceride.

The fatty acid compounds of the invention are combined with a cyclodextrin to form a complex. Cyclodextrins are cyclic oligosaccharides which consist of 6, 7 or 8 α(1-4)-linked anhydroglucose units. The α, β, γ-cyclodextrins, which are prepared by, for example, enzymatic starch conversion, differ in the diameter of their hydrophobic cavity and are generally suitable for the inclusion of a large number of lipophilic substances. Any cyclodextrin is preferably unsubstituted.

The topical compositions of the invention comprise at least one unsaturated fatty acid compound and at least one cyclodextrin. The compositions of the invention can therefore comprise one fatty acid compound and one cyclodextrin compound, a plurality of fatty acid compounds with one cyclodextrin compound, a plurality of fatty acid compounds with a plurality of cyclodextrin compounds or one fatty acid compound with a plurality of cyclodextrin compounds.

Any form of cyclodextrin may be used in the invention, e.g. alpha, beta or gamma cyclodextrin. These are commercially available materials. The cyclodextrins of use in the invention can be optionally substituted. Preferred substituents include alkyl groups, hydroxyalkyl groups and acyl groups For reviews on pharmaceutical acceptable cyclodextrin derivatives see: K.Uekama et al in J. Inclution Phenomena and Macrocyclic Chemistry (2006) 56:page 3-8,T.Loftsson et al. in Am. J.Drug Deliv. (2004)2:page 261-275 and J.Szejtli in J. Inclution Phenomena and Macrocyclic Chemistry (2005)52:1-11. Unless otherwise stated the term alkyl group used herein means C1-10 alkyl groups, preferably C1-4 alkyl groups. This definition applies. to groups containing alkyl groups, e.g. acyl groups.

Among preferred cyclodextrins according to the present invention are unsubstituted alpha-, beta- or gamma-cyclodextrins and methyl or hydroxypropyl substituted derivatives thereof. The most preferred cyclodextrins are the unsubstituted cyclodextrins: alpha-cyclodextrin, beta-cyclodextrin and gamma-cyclodextrin.

The term complex is used here to designate that the fatty acid compound is associated with the cyclodextrin through some form of intermolecular non-covalent bonds. These bonds include normally relatively weak bonds like hydrophobic interactions. The fatty acid compound in the cyclodextrin complex is normally located within the core of the cyclodextrin molecule but could also be associated with other parts of the molecule.

It will be appreciated that where there is more than one fatty acid compound present, there can be more than one cyclodextrin complex formed. A more preferred aspect of the present invention relates to compositions comprising cyclodextrin complexes with two or more fatty acid compounds. It is also within the scope of the invention for a mixture of cyclodextrins to be used, e.g. beta and gamma cyclodextrin or derivatives thereof. The most preferred combinations include EPA ethyl ester/unsubstituted cyclodextrin and DHA ethyl ester/ unsubstituted cyclodextrin mixtures and mixtures of various omega-3 fatty acid glycerides/unsubstituted cyclodextrin.

As noted above, in order to provide a physiologically meaningful dose, a significant amount of fatty acid compound needs to be applied to the skin surface. In a preferred embodiment of this invention, the fatty acid compound/cyclodextrin complexes comprise a relatively high amount of fatty acid compound in the complex. This ensures that high amounts of fatty acid compound are delivered.

The ideal weight ratio depends upon the total composition of oil, the amount of oil present in the formulation and finally on the exact cosmetic/dermal formulation.

The ratio between cyclodextrin (total) and marine fatty acid compound(s) (total) may range from 10:1 to 1:10, e.g. 5:1 to 1:5, preferably 3:1 to 1:3.

The weight ratio between cyclodextrin (total) and unsaturated fatty acid compound (or unsaturated compounds total) may also be more than 1:1, more preferably more than 1:3 , even more preferably more than 1:4, most preferably more than 1:5, especially more than 1:10.

This ratio is especially important for dosage forms for pharmaceutical use as the ratio maximises the amount of active unsaturated fatty acid component present and therefore allows the formation of more concentrated dosage forms.

For cosmetic applications, the concentration of the fatty acid component is still important and it is still preferred if the ratio is as described above. It will be appreciated however that broader ratios can be employed in cosmetic applications and from a stability point of view, a higher level of cyclodextrin may improve long term stability, especially once any cosmetic has been exposed to the air.

For cosmetic applications therefore the ratio between fatty acid compounds (total) or unsaturated compounds (total) and cyclodextrin (total) may also be more than 1:1, more preferably more than 1:3 , even more preferably more than 1:4, most preferably more than 1:5, such as up to 1:10 or more, e.g. up to 1:20.

Viewed from another aspect the invention provides a topical cosmetic composition comprising at least one cyclodextrin complex, e.g. alpha or beta cyclodextrin complex, of a marine unsaturated fatty acid or derivative thereof wherein the weight ratio of cyclodextrin to marine fatty acid or derivative thereof is between 1:10 and 20:1.

The weight ratio between cyclodextrin (total) and omega-3 fatty acid compound (or compounds total) may also be more than 1:1, more preferably more than 1:3 , even more preferably more than 1:4, most preferably more than 1:5, especially more than 1:10.

Surprisingly, this relatively high amount of fatty acid compound versus amount of cyclodextrin has been found still to result in a stable fatty acid compound composition which resists oxidation. Highly preferred ratios range from cyclodextrin to marine fatty acid or derivative thereof of 10:1 to 1:2, especially 4:1 to 1:1, e.g. 3:1 to 1:1.

Whilst ideally all unsaturated fatty acid compound present will be complexed, there is a possibility that some unsaturated fatty acid compounds remain uncomplexed. Without wishing to be limited by theory, it will be appreciated that as there is an excess of fatty acid compound present (and in some embodiments, a large excess of fatty acid compound) relative to cyclodextrin, some fatty acid compound may not be complexed with the cyclodextrin

The complexes of the invention can be water soluble or not water soluble. It is preferred if complexes of the invention are sparingly water soluble or not water soluble, i.e. have a water solubility of less than 18 g/L, preferably less than 10 g/L, such as less than 1.5 g/L.

Complexes of fatty acid compounds with cyclodextrin are known in the art and hence techniques for their formulation are also known. Thus, the cyclodextrin complexes with fatty acid compounds can be prepared using state of the art techniques for preparation of cyclodextrin complexes. Typical methods include for example formation of the complex in water, in mixture of water and organic solvents or water-free organic solvents at ambient temperatures. Suitable organic solvents include methanol, ethanol, isopropanol, acetone, DMSO, DMF and acetonitrile. The cyclodextrin complex with fatty acid compound can then be isolated by filtration, evaporation or freeze drying.

A convenient method for the synthesis of the desired fatty acid cyclodextrin complex involves the use of water as a solvent for the cyclodextrin which can then be mixed with the fatty acid compound e.g. in the form of an ester. The complex forms and can be separated, e.g. by filtration and washed.

An alternative technique utilises organic solvent, e.g. an aqueous alcohol, in which both fatty acid compound and cyclodextrin can be refluxed. The formed complex can be collected by filtration after cooling.

The complex of fatty acid compound with cyclodextrin is a solid. Most preferably the solid is in the form of a powder. In a highly preferred embodiment, the material is a crystalline solid. It should not be an oily material.

As an alternative to the use of cyclodextrins, or optionally as well as their use, the invention covers the use of calixarenes to form complexes with the fatty acid compounds, Calixarenes are macrocyclic compounds capable of assuming a basket (or “calix”) shaped conformation. They are formed from p-hydrocarbyl phenols and formaldehyde and the term applies to a variety of compounds derived by substitution of the hydrocarbon cyclo {oligo[(1,3-phenylene) methylene]}.

A discussion of the use of calixarenes in complexation of amphiphilic molecules can be found in Nannelli et al, Molecular Crystals and Liquid Crystals (2001), 367 621-630.

This forms a further aspect of the invention which therefore provides a topical composition, e.g. pharmaceutical or cosmetic composition, comprising at least one calixarene complex of an unsaturated fatty acid or derivative thereof.

The ratios discussed above in relation to cyclodextrin complexation also apply mutatis mutandis to calixarene complexes.

In addition to fatty acid compound complexes with cyclodextrin or the like, the topical compositions of the invention may comprise substances normally found in topical compositions, e.g. for pharmaceutical or cosmetic use. Such components include typically water, various oils, stabilizers, surfactants, antioxidants, alcohol, perfume and preservatives.

A further preferred aspect of the present invention is to include antioxidants, in particular in cosmetic formulations. The antioxidants can be both synthetic antioxidants and natural antioxidants. The cosmetic composition might comprise of one antioxidant or a mixture of antioxidants. The most preferred antioxidants are antioxidants and mixtures of antioxidants listed by INCI (see below). The most preferred single compound antioxidants, are ascorbic acid and derivatives thereof, tocopherols, various polyphenols like for example luteolin, catechin and carnosol and metal chelators like EDTA and citric acid. Among the most preferred antioxidants are natural components and mixtures thereof; especially compounds and extracts from plants, fruits, berries and animals. Among the most preferred antioxidants to be incorporated into the cosmetics comprising cyclodextrin-encapsulated omega-3 fatty acids are extracts of berries like for example cloudberry, lingonberry, sea buckthorn, brambleberry, blueberry, and cranberry. One preferred aspect of the present invention is to use extracts from plants, fruits, berries and animals grown north of the 62^{nd} parallel. Animal sources for such antioxidants include for example reindeer and moose. The concentration of antioxidants in the compositions according to the present invention is below 1 weight-%, more preferably below 0.5 weight-% and typically around 0.1 weight-%

It will be appreciated that any antioxidant used should not damage the skin and should therefore be approved for use in a cosmetic or pharmaceutical. The cosmetics industry has a “hot list” of antioxidant components which are not appropriate for use on cosmetics and the skilled man would be aware of that.

Preferred antioxiants include flavenoids such as anthocyanidins, catechins, flavones and flavonones. Preferred compounds include quercetin, romarin extract, hesperitin, gallates, tannins, lignans, lignins and other plant extracts.

Especially preferably the anti-oxidant is a polyphenolic antioxidant, e.g. resveratrol, enterodiol, chlorogenic acid, quercetin, (+)-catechin, genisten, hesperetin, cyanidin, casuarictin, procyanidin trimer, rosmarinic acid, pelargonidin, delphinidin, guibourtinidin, fisetinidin, robinetinidin, apigeninidin, luteolinidin, isoquercetin, ellagic acid or a mixture thereof.

Flavonoids of interest as antioxidants include Epicatechin gallate (ECG), Epigallocatechingallate (EGCG), Quercetin (QR), Fisetin (FS), Epigallocatechin (EGC), Catechin (CT), Epicatechin (EC), Rutin (RT), Morin (MR), Kaempherol, Hesperetin (HT), Hesperidin (HD), Naringenin (NG), Naringin (N), Hydroxycinnamic Acids, Caffeic acid (CFA), Chlorogenic acid (CGA), Ferulic acid (FRA), and p-Coumaric acid (CMA).

It will be appreciated therefore that naturally occurring antioxidants are therefore preferred, especially naturally occurring polyphenolic antioxidants.

A cosmetic composition of the invention might also in addition to fatty acid compound cyclodextrin complexes comprise various cosmetic ingredients. A list of such ingredients is available from the International Nomenclature of Cosmetics (INCI). This list including further information regarding useful cosmetic ingredients is available at: www.ec.europe.eu.

Suitable cosmetic preparations include any cream, gel, emulsion, lotion, paste, soap, powder, mousse, etc used in normal cosmetic formulations. Products include body lotion, gels, toothpaste, balms, shower products such as shampoos and conditioner, shower gels, skin creams, moisturisers and the like. In a further preferred embodiment the invention provides a wet wipe impregnated with the composition or complex of the invention.

This use of the compounds of the invention is new and forms another aspect of the invention which therefore provides a wet wipe comprising at least one cyclodextrin complex of a marine unsaturated fatty acid or derivative thereof wherein the weight ratio of cyclodextrin to marine fatty acid or derivative thereof is between 1:10 and 20:1.

A further preferred embodiment involves the use of the composition of the invention as a sunscreen. The inventors believe that unsaturated fatty acids and deriviatives thereof will provide valuable sun protection. Suitably formulated with standard sunscreen excipients, the complex of the invention can provide a sun screen effect, especially in conjunction with other sunscreen agents such as metal oxides.

The compounds of the invention are preferably formulated as pharmaceutically acceptable compositions. The phrase “pharmaceutically acceptable”, as used in connection with compositions of the invention, refers to molecular entities and other ingredients of such compositions that are physiologically tolerable and do not typically produce untoward reactions when administered to a mammal (e.g. human). Preferably, as used herein, the term “pharmaceutically acceptable” means approved by a regulatory agency of the Federal or a state government or listed in the U.S. Pharmacopoeia or other generally recognized pharmacopoeia for use in mammals, and more particularly in humans.

Pharmaceutical compositions of the invention will typically comprise a carrier. The term “carrier” applied to pharmaceutical compositions of the invention refers to a diluent, excipient, or vehicle with which an active compound is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by E. W. Martin, 18th Edition, incorporated by reference.

The compositions of the invention are proposed for use in the treatment of, inter alia, skin disorders. By treating or treatment is meant at least one of:

(i). preventing or delaying the appearance of clinical symptoms of the disease developing in a mammal;
(ii). inhibiting the disease i.e. arresting, reducing or delaying the development of the disease or a relapse thereof or at least one clinical or subclinical symptom thereof, or
(iii). relieving or attenuating one or more of the clinical or subclinical symptoms of the disease.

The benefit to a subject to be treated is either statistically significant or at least perceptible to the patient or to the physician. In general a skilled man can appreciate when “treatment” occurs.

Prophylactic treatment means treating subjects who are at risk of developing a disease in question.

The compounds of the invention can be used on any animal subject, in particular a mammal and more particularly to a human or an animal serving as a model for a disease (e.g., mouse, monkey, etc.).

A “pharmaceutically acceptable excipient” means an excipient that is useful in preparing a pharmaceutical composition that is generally safe, non-toxic and neither biologically nor otherwise undesirable, and includes an excipient that is acceptable for'veterinary use as well as human pharmaceutical use. A “pharmaceutically acceptable excipient” as used in the present application includes both one and more than one such excipient.

The pharmaceutical formulations of the present invention can be liquids that are suitable for topical administration, for example creams, salves, gels, ointments or solutions, suspensions, emulsions, or other forms suitable for administration by the topical, e.g. transdermal route. Ideally the topical compositions of the present case will be applied to the external skin surface.

The compositions of the invention can be administered for immediate-, delayed-, modified-, sustained, or controlled-release applications. Pharmaceutical compositions can be prepared by mixing a therapeutically effective amount of the active substance with a pharmaceutically acceptable carrier that can have different forms, depending on the way of administration.

Pharmaceutical compositions can be prepared by using conventional pharmaceutical excipients and methods of preparation. The compositions of the invention may contain from 0.01 to 99% weight of the complex. The topical composition of the invention will preferably comprise up to 50 wt %, e.g. up to 40 wt %, preferably up to 30 wt %, such as up to 20 wt % of the fatty acid/cyclodextrin complex. Most preferably, the topical composition of the invention comprises 0.1% to 10% (weight) of at least one fatty acid compound in the form of a complex with cyclodextrin.

Most preferably, the topical compositions of the invention contain 0.1% to 10% (weight) omega-3 fatty acid compound , preferably 0.2% to 9% (weight), more preferably 0.3% to 8.5%, especially 0.4% to 8%, most especially 0.5% to 8%.

In further preferred embodiments, the topical compositions of the invention contain 0.01% to 1% (weight) omega-3 fatty acid compound , preferably 0.02% to 0.75% (weight), more preferably 0.05% to 0.5%, especially 0.025% to 0.25%.

A therapeutically effective amount of the compound of the present invention can be determined by methods known in the art. The therapeutically effective quantities will depend on the age and on the general physiological condition of the patient, the route of administration and the pharmaceutical formulation used. The therapeutic doses will generally be between about 0.2 and 2000 mg/day and preferably between about 0.5 and 1500 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

The composition of the invention will preferably enhance skin properties or treat or prevent of skin diseases. Preferably, the topical compositions of the invention will have at least one of the following effects on the skin: anti-aging effect, increasing collagen synthesis in the human skin, improvement of skin hydration, increased skin elasticity, improve smoothening of the skin, increase the tensile properties of the skin, increased firmness, reduction of formation of fine lines in the skin, reduction of skin wrinkles, increasing skin smoothness, treatment or prophylaxis of cellulite.

Diseases of the skin against which the compositions of the invention might provide benefit include acne and dermatitis.

The topical compositions of the invention are regarded as stable. Preferably they can be stored for at least 6 weeks without degrading, more preferably the compositions can be stored for at least 3 months, preferably at least 6 months, especially at least 1 year.

In a further embodiment excipients used in the formation of the topical compositions of the invention can also be complexed with cyclodextrin. In particular tocopherol can be complexed with cyclodextrin and used as an excipient. The invention is further illustrated by the following non-limiting examples:

### Intermediate 1

EPA and DHA-ethyl ester complex with beta-cyclodextrin. Ratio 1:1 fatty acid derivative and cyclodextrin
Beta-cyclodextrin(5.0 grams) was dissolved in water (200 ml). The solution was cooled to room temperature. A mixture of EPA- and DHA-ethyl ester (Omacor®) (5.0 grams) was added. The mixture was stirred under an atmosphere of argon in the dark for 3 days. The product was isolated by centrifugation and dried. The product was a white powder.

### Intermediate 2.

Cod liver oil complex with beta-cyclodextrin. Ratio 1:1 (weight) cod liver oil to cyclodextrin .

Beta-cyclodextrin (20 grams) and water (20 ml) were added into a mortar. The mixture was ground for 2 minutes. Cod liver oil (Mollers Omega-3 tran from Moller, Oslo, Norway) (20 grams) was added and the mixture was ground for 30 minutes. The product was dried in vacuum at 60° C. for 5 hours. A white non-greasy powder was isolated.

### Intermediate 3

Tocopherol complex with beta-cyclodextrin. Ratio 1:5(weight) tocopherol to cyclodextrin.

Tocopherol (1.0 gram) was dissolved in 2-propanol (50 ml). Beta-cyclodextrin (5.0 gram) was added and the mixture was stirred at reflux temperature for 30 minutes. The mixture was evaporated and dried and further dried at high vacuum over night. The title product was isolated as a white powder.

### Intermediate 4

Salmon oil complex with beta-cyclodextrin (25% oil)

Beta cyclodextrin (50 grams) and water (35 ml) were mixed in a mortar grinder for 5 minutes. Salmon oil (Xalar™ Refined Salmon oil from Marine Harvest, Hjelmeland, Norway)(17 grams) was added and the mixture was kneaded for 10 minutes. The product was dried by freeze-drying for 40 hours. A white, non-greasy, odourless and tasteless powder was isolated.

### Intermediate 5

Salmon oil complex with beta-cyclodextrin (20% oil)

The product was prepared as described in Intermediate 4 using beta cyclodextrin (60 grams), water (42 ml) and Salmon oil (Xalar™ Refined Salmon oil from Marine Harvest, Hjelmeland, Norway)(15 grams). A white, non-greasy, odourless and tasteless powder was isolated.

### Intermediate 6

Salmon oil complex with beta-cyclodextrin (15% oil)

The product was prepared as described in Intermediate 4 using beta cyclodextrin (51 grams), water (36 ml) and Salmon oil (Xalar™ Refined Salmon oil from Marine Harvest, Hjelmeland, Norway)(9 grams). A white, non-greasy, odourless and tasteless powder was isolated.

### Intermediate 7

Salmon oil complex with beta-cyclodextrin (10% oil)

The product was prepared as described in Intermediate 4 using beta cyclodextrin (54 grams), water (38 ml) and Salmon oil (Xalar™ Refined Salmon oil from Marine Harvest, Hjelmeland, Norway)(6 grams). A white, non-greasy, odourless and tasteless powder was isolated.

### Intermediate 8

Cloudberry extract

Cloudberry picked in Norwegian mountains (Rendalen, appr. 1000 meters above see level) (20 grams) were stirred together with methanol (100 ml) at 50 degrees centigrade for 1 hour. The mixture was cooled, filtered and evaporated. A yellow semisolid material was isolated (3.1 grams)

### Intermediate 9

Green tea extract

Green tea (Alwazah from George Payne & Co.) (20 g) was added to water (200 ml) at boiling point. The mixture was stirred for 5 minutes and filtered. The aqueous phase was isolated as a green-brown opaque solution.

### Intermediate 10

Apricot extract

Sun dried apricots (Boyraz from Turkey) (60 grams) were cut in pieces, methanol (100 ml) was added and the mixture was stirred for 10 minutes at boiling temperature. The methanol solution was filtered and evaporated leaving a yellow viscous oil. Yield : 10.9 grams

### Intermediate 11

Fish oil alpha-cyclodextrin complex

Alpha-cyclodextrin (10 grams) and water (5 ml) were kneaded using a mortar and pestle for 5 minutes. Fish oil (EPAX 6000 TG) (3 grams) were added and the mixture was kneaded for another 10 minutes. The material was dried overnight in vacuum. The title compound was isolated as a white solid material. (Yield : 13 grams)

### Intermediate 12

Fish oil gamma-cyclodextrin complex

Gamma-cyclodextrin (10 grams) and water (5 ml) were kneaded using a mortar and pestle for 5 minutes. Fish oil (EPAX 6000 TG) (3 grams) were added and the mixture was kneaded for another 10 minutes. The material was dried overnight in vacuum. The title compound was isolated as a white solid material. (Yield : 13 grams)

### Intermediate 13

Juniper extract

Juniper (grown at Nordstrand, Oslo, Norway) (20 grams) were cut in pieces, methanol (100 ml) was added and the mixture was stirred for 10 minutes at boiling temperature. The methanol solution was filtered and evaporated leaving a green solid material. Yield : 400 mg

### Intermediate 14:

Salmon oil complex with beta-cyclodextrin. Ratio 1:3 (weight) salmon oil to cyclodextrin.

Beta cyclodextrin (750 grams) and water (750 ml) were mixed in a kitchen kneader for 15 minutes. Salmon oil (Xalar™ Refined Salmon oil from Marine Harvest, Hjelmeland, Norway)(250 grams) was added and the mixture was kneaded for 30 minutes. The product was dried by freeze-drying for 60 hours. A white, non-greasy, odourless and tasteless powder was isolated.

### EXAMPLE 1

Body lotion comprising 1% cod liver oil cyclodextrin complex.

Cod liver oil beta cyclodextrin complex (Intermediate 2) (1 gram) was added to a preformed body lotion emulsion (99 grams) used volumetic mixturing of the components.

The ingredients in the preformed body lotion were water, paraffinum, sorbitol, petroleum, glycerine, cetearyl alcohol, ceteareth-15, glycerol stearate, aroe, barbadensis, ethylhexyl stearate, dimethicone, panthenol, perfume, cinnamyl alcohol, citronellol, limonene, alpha-isomethyl ionone, geraniol, hydroxycitronellal, butylphenyl methylpropionate, linalool, citric acid, benzyl alcohol, PPG-2 methyl ether, 2- bromo-2-nitropropane-1,3-diol, iodopropynyl butylcarbamate and deceth-8.

### EXAMPLE 2

Body lotion comprising 5% cod liver oil cyclodextrin complex.

The product was prepared as described in Example 1 using cod liver oil (5 grams) and preformed body lotion (95 gram).

### EXAMPLE 3

Body lotion comprising 10% cod liver oil cyclodextrin complex

The product was prepared as described in Example 1 using cod liver oil (10 grams) and preformed body lotion (90 grams)

### EXAMPLE 4

Body lotion comprising 10% cod liver oil cyclodextrin complex and 1% tocopherol cyclodextrin complex.

The product was prepared as described in Example 1 using Intermediate 2 (10 gram) and Intermediate 3 (1 gram) and preformed body lotion (89 grams)

### EXAMPLE 5

Body lotion comprising 10% cod liver oil cyclodextrin complex and 0.5% tocopherol cyclodextrin complex.

The product was prepared as described in Example 1 using Intermediate 2 (10 gram) and Intermediate 3 (0.5 gram) and preformed body lotion (89.5 grams)

### EXAMPLE 6

Stability of body lotion comprising cod liver oil.

Products prepared according to Example 1 and Example 5 were kept in closed containers for 6 weeks. The products were evaluated for cosmetic use on the skin.

No fishy odour was observed.

### EXAMPLE 7

Body lotion comprising 0.2% salmon oil cyclodextrin complex and cloudberry extract.

The product was prepared as described in Example 1 using salmon oil cyclodextrin complex (intermediate 4) (200 milligrams) and cloudberry extract (intermediate 8) (0.300 milligram) and preformed body lotion (99.5 grams)

### EXAMPLE 8

Gel comprising 0.8% salmon oil cyclodextrin complex and cloudberry extract

Salmon oil beta cyclodextrin complex (Intermediate 5) (800 milligram) and cloudberry extract (intermediate 8) (400 mg) were added to a preformed aloe vera gel (98.8 grams) using volumetic mixturing of the components.

The ingredients in the preformed gel were water, Aloe Barbadensis leaf Juice,PEG-33,PEG-8 dimethicone,PEG-14, acrylates/C10-30 alkyl acrylate crosspolymer, disodium EDTA, DMDM, hydantoin, benzophenone-4, parfum, PEG-40, hydrogenated castor oil, benzyl salicylate, butylphenyl methylpropional, vitronellol, hydroxyisohexyl 3-cyclohexenecarboxyaldehyde, linalool, CI15985 and CI 74160.

### EXAMPLE 9

Tooth paste comprising 1.6% salmon oil cyclodextrin. complex .

Salmon oil beta cyclodextrin complex (Intermediate 6) (800 milligram) was added to a preformed toothpaste (49.1 grams) using volumetic mixturing of the components.

The ingredients in the preformed tooth paste were water, sorbitol, hydrated silica, propylene glycol, cellulose gum, sodium lauroyl sarcosinate, disodium pyrophosphate, tetrasodium pyrophosphate, sodium monofluorophosphate, sodium saccharin, allantoin, sodium propylparaben, sodium methylparaben, menthe arvensis cornmint, limonene, aroma, CI 77891.

### EXAMPLE 10

Balm comprising 1% salmon oil cyclodextrin complex and green tea extract

Salmon oil beta cyclodextrin complex (Intermediate 6) (1.0 gram) and green tea (intermediate 9) (2 ml) were added to a preformed balm 97.5 grams) used volumetic mixing of the components.

The ingredients in the preformed balm were water, cetearyl alcohol, glycerine, cetrimonium chloride, parfum, panthenol, citric acid, amodimethicone, C11-15 pareth-7,C12-16 pareth-9, trideceth-12, 2-bromo-2-nitropropane-1,3-diol.

### EXAMPLE 11

Shower soap comprising 1.0% salmon oil cyclodextrin complex .

Salmon oil beta cyclodextrin complex (Intermediate 4) (1.0 gram) was added to a preformed shower soap (99 grams) used volumetic mixing of the components.

The ingredients in the preformed shower soap were water, sodium laureth sulphate, sodium chloride, sodium cocoamphoacetate, parfum, phenoxyethanol, benzoic acid, dehydroacetic acid, citric acid, tetrasodium iminosuccinate, propylene glycol, propyl gallate, CI16255 and CI 19140.

### EXAMPLE 12

Shampoo comprising 1% salmon oil cyclodextrin complex

Salmon oil beta cyclodextrin complex (Intermediate 5) (1 gram) was added to a preformed shampoo (99 grams) used volumetric mixing of the components.

The ingredients in the preformed shampoo were water, sodium laureth sulphate, sodium chloride, sodium cocoamphoacetate, PEG-4 rapeseedamide, glycerine, glycol distearate, phenoxyethanol, benzoic acid, dehydroacetic acid, cocamide MEA, Laureth-10, citric acid, propylene glycol, propyl gallate, parfum, panthenol, tetrasodium iminodisuccinate, formic acid, CI 19140 and CI 16255

### EXAMPLE 13

Body lotion comprising 1% fish oil alpha-cyclodextrin complex and apricot.

Fish oil alpha-cyclodextrin complex (Intermediate 11) (1 gram) and apricot extract (Intermediate 10) (300 mg) were added to a preformed body lotion emulsion (99 grams) by volumetric mixing of the components.

### EXAMPLE 14

Body lotion comprising 1% fish oil gamma-cyclodextrin complex and juniper extract.

Fish oil gamma-cyclodextrin complex (Intermediate 12) (1.2 gram) and juniper extract (Intermediate 13) were added to a preformed body lotion emulsion (99 grams) by volumetric mixing of the components.

### EXAMPLE 15

Skin cream comprising 0.1% salmon oil cyclodextrin complex.

Salmon oil beta cyclodextrin complex (intermediate 14) was formulated into a low fat skin lotion. The skin lotion contained water, ethylhexyl cocoate, glycerin, polyacrylamide, C13-14 isoparaffin, laureth-7, phenoxyethanol, benzyl alcohol, potassium sorbate, tocopherol, sodium citrate, citric acid and disodium EDTA

### EXAMPLE 16

Skin cream comprising 1% salmon oil cyclodextrin complex.

The product was prepared as described in Example 15 using intermediate 14.

### EXAMPLE 17

Stability of body lotion comprising salmon oil.

Products prepared according to Example 15 and Example 16 were kept in petri dishes at 40° C. for two weeks. Primary and secondary lipid oxidation products were measured using SafTest® PeroxySafe™ and SafTest® AlkalSafe™ rapid test kits from MP Biomedicals (Solon, Ohio, USA). No oxidation was detected in the creams after storage.

### EXAMPLE 18

Wet wipe (alcohol based) comprising omega-3 alpha-cyclodextrin, omega-3 gamma-cyclodextrin and apricot extract

Omega-3 alpha-cyclodextrin (Intermediate 11) (50 mg), omega-3 gamma-cyclodextrin (Intermediate 12) (50 mg) and apricot extract (Intermediate 10) (80 mg), body lotion (same as used in Example 1) (5 grams) and ethanol (15 ml) were heated to appr. 50 decrees centigrade and stirred for 2 minutes. The solvent mixture was cooled to room temperature. A paper towel (dry weight 2.4 grams, size 20 cm times 20 cm) was impregnated with the solvent mixture. The wet wipe was packed in an airtight plastic bag.

### EXAMPLE 19

Wet wipe (non-alcohol based) comprising gel with salmon oil beta-cyclodextrin and cloudberry extract

A paper towel (dry Weight 2.4 grams, size 20 cm times 20 cm) was impregnated with the gel from Example 8 (6 grams). The wet wipe was packed in an airtight plastic bag.

1. A topical composition, comprising at least one cyclodextrin complex of a marine unsaturated fatty acid or derivative thereof wherein the weight ratio of cyclodextrin to marine fatty acid or derivative thereof is between 1:10 and 10:1.

2. A composition as claimed in claim 1 wherein the weight ratio of cyclodextrin to marine fatty acid or derivative thereof is between 1:10 and 1:1.

3. A composition as claimed in claim 1 wherein the weight ratio of cyclodextrin to marine fatty acid or derivative thereof is 4:1 to 1:2.

4. A composition as claimed in claim 1 wherein the cyclodextrin is alpha-cyclodextrin, beta-cyclodextrin or gamma-cyclodextrin.

5. A composition as claimed in claim 1 wherein the cyclodextrin is beta-cyclodextrin.

6. A composition as claimed in claim 1 wherein the marine unsaturated fatty acid or derivative thereof is derived from fish oil, e.g. salmon oil or cod liver oil.

7. A composition as claimed in claim 1 wherein the marine unsaturated fatty acid or derivative thereof comprises DHA or EPA or a derivative thereof.

8. A composition, preferably a cosmetic composition, as claimed in claim 1 further comprising at least one antioxidant.

9. A composition as claimed in claim 8 wherein the antioxidant is a polyphenolic antioxidant.

10. A composition as claimed in claim 1 being a sunscreen, body lotion, toothpaste or cream.

11. A topical composition comprising at least one cyclodextrin complex of a marine unsaturated fatty acid or derivative thereof wherein the weight ratio of cyclodextrin to fatty acid or derivative thereof is between 1:10 and 10:1 for enhancing skin properties or for the treatment or prophylaxis of skin diseases.

12. (canceled)

13. A method of enhancing skin properties comprising topically applying a to the skin of a patient a topical composition as claimed in claim 1.

14. A method of treatment or prophylaxis of skin diseases comprising applying to the skin a patient a topical composition as claimed in claim 1.

15. A process for the manufacture of a topical composition as claimed in claim 1 comprising forming a cyclodextrin complex with a marine unsaturated fatty acid or derivative thereof and preparing a topical composition thereof.

16. A topical composition, comprising at least one cyclodextrin complex of an omega-3 fatty acid or derivative thereof.

17. A topical cosmetic composition comprising at least one cyclodextrin complex, of marine unsaturated fatty acid or derivative thereof wherein the weight ratio of cyclodextrin to marine fatty acid or derivative thereof is between 1:10 and 20:1.

18. A wet wipe comprising at least one cyclodextrin complex of a marine unsaturated fatty acid or derivative thereof wherein the weight ratio of cyclodextrin to marine fatty acid or derivative thereof is between 1:10 and 20:1.

19. A topical cosmetic composition of claim 17 wherein the at least one cyclodextrin complex comprises an alpha or beta cyclodextrin complex.
